(19) United States
(12) Patent Application Publication
(10) Pub. No.: US 2016/0129215 A1
(43) Pub. Date: May 12, 2016

(54) Method of Training a Living Body to Not React to Substances

(71) Applicant: Dawn Nowlin, Leesburg, VA (US)

(72) Inventor: Dawn Nowlin, Leesburg, VA (US)

(21) Appl. No.: 14/996,202

(22) Filed: Jan. 14, 2016

Related U.S. Application Data
(63) Continuation-in-part of application No. 14/004,257, filed on Sep. 10, 2013, now abandoned.

(51) Int. Cl. A61M 21/00; A61H 23/02; A61N 1/36

(52) U.S. Cl. CPC A61M 21/00; A61N 1/36014; A61H 23/02; A61M 2021/0022; A61M 2021/0072

(57) ABSTRACT

A method of conditioning a living body of a patient to associate the unconditioned stimulus (US) of decreased sympathetic nerve activity SNA) with the conditioned stimulus (CS) of a representation of an offending agent to cease or reduce defensive reactions or symptoms. The US involves non-invasive, transcutaneous sensory stimulation of the sympathetic ganglia, which is paired with the CS of digital audio representations of offending substances to modify pathologically conditioned. reflexes of various systems involved. in a reaction. In addition, a patient's allergies or sensitivities are treated by using digital representations, preferably provided via a computer, to represent the actual substances in order to engage the multimodal functioning of the brain. Sensory stimulation is used in conjunction with the digital audio signals to condition the body to respond more appropriately to the substance.

This is a continuation-in-part application of U.S. patent application Ser. No. 14/004,257 filed on Sep. 10, 2013, and priority is claimed thereto.

### FIELD OF THE INVENTION

The present invention is a method of conditioning a living body of a patient to associate a decrease in sympathetic nerve activity from sensory stimulation of the sympathetic ganglia as the primary stimulus digital audio representations of offending substances as the secondary stimulus to modify defensive physiological reactions of bodily systems; to ultimately reduce negative reactions to such substances. In addition, the method of the present invention takes advantage of multimodal functioning of the brain, thus using audio digital representations of the substance being treated.

### BACKGROUND OF THE PRESENT INVENTION

Allergies and Sensitivities

Allergies and sensitivities are the result of bodily systems reacting inappropriately to harmless, often naturally occurring substances, which can affect virtually every part of the body. These reactions are abnormal.

A large percentage of reactions to harmless substances do not involve the immune system.

Increasingly, evidence is showing that mast cells, which play a central role in the allergic response, can actually be activated directly, by non-alleric triggers. Such triggers may include environmental chemicals, foods, drugs, odors, temperature changes, physical or emotional stimuli and stress. Mast cells are found in tissues throughout the body and it is well established that there is an anatomic association between mast cells and nerves in most tissues. Such reactions that do not involve the immune system are referred to as sensitivities. Sensitivities are an understudied area with no treatment options other than avoidance. The symptoms of sensitivities may be as pronounced and can even he as severe as those of true allergies, with no immune involvement. It has only been recently recognized that sensitivity-related illness (SRI) may involve various organ systems and evoke wide ranging physical or neuropsychological manifestations. The inventor has concluded that the body's response to a substance may depend on the interpretation or perception of that substance. If the immune system or other systems in the body have developed a perception of a substance as being harmful or a threat, this may result in a broad range of symptoms caused by defensive physiological behavior. If the substance is benign, an inappropriate reaction is the result of an errant perception or a pathologically developed association with the substance, leading to sensitivity reactions.

This invention is not based on treating the immune system, but instead approaches the physiological reaction allergies and sensitivities by addressing the perceptual error or pathologically conditioned reflex. The invention takes advantage of adaptive mechanisms of the body utilizing a conditioning method that enables the human brain and cognitive processes to adapt to the external milieu.

The first known study on the phenomenon of conditioning was in 1902, by Ivan Pavlov, demonstrating that conditioned reflexes may be learned either by repetitive stimuli or by associating two stimuli. The conditioning process causes a learned behavior that responds to the associated stimulus based on the newly interpreted meaning of that stimulus. Pavlov concluded that a connection was made in the nervous system linking an environmental stimulus to an unconditioned reflex, transforming the reflex into a conditioned reflex, activated by an external stimulus. This invention utilizes a non-invasive, transcutaneous sensory stimulus to the sympathetic ganglia as the primary stimulus, which activates an unconditioned reflex of decreased Sympathetic Nerve Activity (SNA), and contextual linguistic audio digital signals of an offending agent as the secondary stimulus to create a new association or interpretation of the treated substance. The process which Pavlov described as a formation of conditioned reflexes is essentially a translation of messages from a psychological sign system into messages of a system of somatic signs and vice versa. With this translation, a connection develops between the psychological and the somatic levels. The stimulus to the body and use of contextual representations for cognitive processes can he integrated and, in fact, are connected through a temporal pairing.

The Immune System and Classical Conditioning

Studies have shown that altered immune function can be induced through classical behavioral conditioning (Elenkov et al, 2000; Ader et al. 1987; Metalnikov 1926). Elements in the environment may randomly be associated with either stressful stimuli or symptoms being experienced at he time of exposure. This coupling of the two stimuli is learned and stored, and may cause a chronic pattern of reactivity. See Elenkov, I. J., R. L. Wilder, G. P. Chrousos and E. S. Vizi (2000). “The Sympathetic Nerve An Integrative Interface between Two Supersystems: The Brain and the Immune System,” Pharmacological Reviews 52(4): 595-638. Also see Ader R, Grota L J, Cohen N. (1987). Conditioning phenomena and immune function. Ann N Y Acad SM. 1987;496:532-44.

A series of studies 1920's (Metal'nikov et al 1926) provided strong evidence that immune reactions could be conditioned by classical Pavlovian means. The studies demonstrated that conditional reflexes can play a very important role not only in the immune response but also in various ailments. The authors concluded that everything surrounding the patient can act as conditional stimuli, associated with the symptoms being experienced, causing the same disease as the primary cause.

Pain, discomfort sensations and other symptoms can occur not only under the influence of natural causes (viruses, poisoning, etc.) but also by the action various stimuli that associate accidentally during illness. It was concluded that many chronic and nervous diseases (asthma, heart disorders, neurosis, etc.) and paroxysms may occur under the influence of conditioned stimuli which have nothing in common with the essential cause of the disease. The recommended treatment for these conditions caused by classical conditioning was to remove the conditioned stimuli that are created during, illness or other stressful events. Avoidance is the only known method of treatment. This invention's method utilizes the same learning phenomenon to reverse the conditioned reflex by pairing the conditioned stimulus with decreased sympathetic nerve activity as opposed to the increased sympathetic nerve activity which is present during any mode of stress and has also been shown to be active in negative emotions such as fear and anger. See Metal'nikov S, Chorine V (1926) Rôle des réflexes conditionnels dans l'immunité. Ann Inst Pasteur Paris 40;893-900.

The inventor has discovered that the human body can react inappropriately to a vast number of benign substances or stimuli which may he caused by conditioned reflexes. Recent investigations by Icenhour et al (2015) demonstrate that artificially-induced intestinal pain may be paired with visual symbolic stimuli (circles and squares) to form an association resulting in a conditioned reflex. Presentation of the visual cues late reproduced the same intestinal symptoms, thereby supporting Metal'nikov and Chorine's conclusions that the immune system and other bodily systems can be conditioned to random stimuli in the environment. Crucially, these findings provide further evidence for this invention's claim that the symbolic level of cognitive processes is not separate from the immune system or other physiological systems and can be involved in faulty conditioning as well as corrective conditioning processes. See Icenhour, A. L., J.; Benson S.; Schlamann, M.; Hampel, S.; Engler, H.; Forsting, M.: Eisenbruch, S. (2015). “Neural circuitry of abdominal pain-related fear learning and reinstatement in irritable bowel syndrome.” Neurogastroenterol Motil 27: 114-127.

The Sympathetic Nervous System

The traditional view that the immune system is autonomous has been successfully challenged over the last few decades. There is now strong evidence that the brain and the immune system, which are the two major adaptive systems of the body, maintain a bidirectional flow of messages. The primary pathway for the neural regulation of immune function is provided by the sympathetic nervous system (Elenkov at al 2000; Nance et al 2007). In the case of sensitivities, which do not involve the immune system, there is an anatomic association between mast cells and nerves. Thus, the nervous system plays an important role in both allergies and sensitivities.

Studies have shown that non-invasive, transcutaneous, electrical nerve stimulation (TENS) of the sympathetic chain decreases activity of the sympathetic nervous system (Stein et al, 2011). The inventor discovered that percussive stimulation in the area of the sympathetic ganglia produces similar results, providing the unconditioned stimulus for this invention's method—to reverse pathologically conditioned reflexes. Percussive stimulation allows for a non-pharmacological non-invasive approach to educe activity of the sympathetic nervous system. See Elenkov, I. J., R. L. Wilder, G. P. Chrousos and E. S. Vizi., 2000. “The Sympathetic Nerve—An Integrative Interface between Two Supersystems: The Brain and the Immune System.” Pharmacological Reviews 52(4): 595-638. Also see Nance, D M, Sanders, V M. 2007. Autonomic Innervation and Regulation of the immune System (1987-2007). Brain Behav Immun. 2007 August; 21(6): 736-745. Additionally, see Stein C, Dal Lago R, Fferreir J B, Casali K R, Della Mea Plentz R. 2011. Transcutaneous electrical nerve stimulation at different frequencies on heart rate variability in healthy subjects. Autonomic Neuroscience: Basic and Clinical 165 (2011) 205-208.

Multimodal Stimulus Input

Pavlov concluded that in animals there exists only a first system of signals of reality, allowing the brain to receive and analyze stimuli within the organism as well as outside the organism, in humans, there exists both this first level as well as a second level of signals; language or symbols. Words and symbols can function as stimuli in humans, so real and effective, that they can mobilize humans just as a concrete stimulus. Words are symbols, abstractions; the conditioned stimulus can be generalizable.

Studies have shown that neural circuits in certain regions of the brain display common patterns of activation for concrete objects regardless of whether the objects are presented visually, auditorily or verbally (Simanova 2014; Marinkovic et al. 2003; Bright et al. 2004; Kircher et al. 2009; Price 2010). Thus, decoding of semantic information is possible from different stimulus modalities, which also engages the symbolic level of cognitive processes. The preferred modality for this invention is the use of digital signals of auditory linguistic representations for the conditioned stimuli for the treatment. The digital signal used in treatment is initially created from a linguistic representation. This linguistic representation is converted to an audio digital signal and transmitted repeatedly during treatment. The invention engages unconscious processes by transmitting the digital signals at low decibels that are unavailable to conscious perception (subthreshold). Studies have shown it brain activity does index the acquisition of a conditional response to subthreshold stimuli and there is strong evidence for associative learning outside awareness in central nervous system activity (Wong et al 1991, Wong et al 2004). The digital audio signal is transmitted via speaker embedded in a cuff or mat that the patient lies on.

See Simanova I, Hagoort P, Oostenveld R, van Gerven M A. 2014. Modality-independent decoding of semantic information from the human brain. Cereb Cortex, 2014 February;24(2):426-34. See also Bright P, Moss H, Tyler I. K. 2004. Unitary vs multiple semantics; PET studies of word and picture processing. Brain Lang 89:417-432. See as well Markinovic K, Dhond R P, Dale A M, Glessner M, Carr V, Halgren E. 2003. Spatiotemporal dynamics of modality-specific and supramodal word processing. Neuron 38:487-497. See Kircher Sass K, Sachs O, Krach S. 2009. Priming words with pictures: neural correlates of semantic associations in a cross-modal priming task using fMRI. Hum Brain Mapp. 30:4116-4128. See additionally Wong P S, Bemat F, Bunce S, Shevrin H. 1991. Brain indices of nonconscious associative learning. Conscious Cogn. 1991 December;6(4):519-44. Also see Wong P S, Bemat E, Snodgrass M, Shevrin It 2004. Event-related brain correlates of associative learning without awareness. Int J Psychophysiol. 2004 August;53(3):217-31.

By presenting a language-based representation of the offending substance to the patient while simultaneously introducing a sensory stimulus that decreases activity of the sympathetic nervous system, the body associates the unconditioned stimulus with the benign substance and alters its perception, no longer interpreting the substance as harmful. The inventor has found that the substance being treated may be introduced to the subject as an audio digital representation of the substance provided by a computer or other device. The multimodal processes of the brain allows for a semantic interpretation of the substance. The inventor has found that cognitive processes can interpret the representation of the substance used in treatment to allow for a pairing of the two stimuli for the conditioned effect.

### SUMMARY OF THE PRESENT INVENTION

The method of the present invention is as follows: Subject is treated by presenting the digital audio representation which represents the substance the body is inappropriately reacting to, preferably via a computer playing a digitized sound, electrical stimulation from TENS or manual percussion is administered at the location of the sympathetic ganglia. Transcutaneous stimulation of these areas has been shown to decrease activity of the sympathetic nervous system. The simultaneous stimulation of sympathetic ganglia with the cognitive input of the signal forms a new association with the substance thereby modifying the physiological reaction. The present invention is designed to engage the multimodal functioning of the brain primarily through audio signals but may also be through visual textual signals.

The mechanism underlying the effect of the treatment is a conditioned association whereby the substance is “coupled” with decreased sympathetic nerve activity of the stimulus. The modified perception of the harmless substance alters the behavior of the effected system(s). The therapeutic stimulation used in conjunction with the transmission of digital signals conditions the body to alter its perception of a harmless substance. The transmission of digital signals has no effect or therapeutic value to the body. It is only the stimulation of the sympathetic ganglia chain in conjunction with the presentation of the digital representation that allows for a resolve in the pathological conditioned reflex.

The inventor has discovered that the human body can inappropriately react to a vast number of substances including stimuli, such as heat or sunlight. Therefore, a database, maintained by a computer, is used to include a large number of substances. This invention is not intended for use on cases of anaphylaxis or life threatening sensitivities.

Manual or electrical stimulation or a mat capable of providing subcutaneous sympathetic ganglia chain stimulation and audible transmission of sound may be employed by the method of the present invention, in lieu of a cuff. The patient would preferably lay his or her back on the mat. The mat contains a speaker, which is preferably embedded or integrated into the mat, which contains vibratory motors designed for stimulation.

### DETAILED DESCRIPTION OF THE EMBODIMENT(S) OF THE PRESENT INVENTION

The present invention is a method that serves to treat digital audio representations associated with a specific substance in order to ultimately condition the body's natural physiology to accept such specific irritant that initially caused the sensitivity reaction. In one embodiment, sensory stimulation is administered to locations near the sympathetic ganglia, along each side of the spine, with the use of vibratory motor stimulation. This stimulation is utilized to temporarily decrease activity of the sympathetic nervous system. Meanwhile, the digital representation specific to the substance found to cause the irritation is played (and optionally presented in text on the computer monitor). The combination of the cognitive perception of the digital audio signals and the stimulation of the sympathetic ganglia conditions the body to not react to that particular substance. In other words, the different digital signals, provided via a computer, that represent an irritant transmitted at the same time as decreasing sympathetic nerve activity ultimately conditions the various systems to respond without defensive behavior.

The present invention proceed as follows. First, a speaker is positioned in the proximity of the patient (via cuff or embedded into the stimulation mat). It should be noted that the patient also might be referred to as person or subject. In one embodiment, the speaker may be placed on the underside of an arm cuff or embedded into the arm cuff. The arm cuff would then be wrapped around the arm of the patient or positioned in the vicinity of the patient's ear. The cuff interfaces with a computer via a USB port. A digital audio signal is transmitted, preferably via a computer. The signal being transmitted is representative of what the actual potential offending agent is purported to be. This means that the sound literally can be words identifying the purported irritant.

While the signal is transmitted toward the patient, a transcutaneous stimulation is applied to the sympathetic ganglia chain utilizing vibratory motors that run alongside the vertebrae. Additional stimulation may be applied to specific sympathetic ganglia correspondiing to organ systems which present the prevalent symptoms. The stimulation, which reduces activity of the sympathetic nervous system, along with the representation if the substance has the effect of conditioning the patients body to perceive the irritant as neutral, instead of a threat, which in turn will diminish the inappropriate reaction.

The mat that is utilized this present invention is a thin foam mat that encases a series of vibratory motors. The series of vibratory motors are, in one embodiment, arranged in two columns which are each 4 feet (121.92 cm) in length; there is a 3 inch (7.62 cm) separation between the two columns. The series of vibratory motors provides a gentle percussion when activated by the practitioner, via the software program. The mat is placed on a treatment table and the patient lies on the mat so that the vibratory motors make contact at the location medial to the paraspinal muscles, specifically between the spine and the paraspinal muscles of the patient. When activated, the device stimulates the sympathetic ganglia chain areas with a gentle percussion. The percussion occurs while the sound is transmitted toward the body of the patient, as described above. And thus, it would be preferred that in the mat is the speaker, located at the top of the mat (near where the patient's head rests) that transmits the digital audio signals. The mat replaces both the need for the positioning of the speaker in the arm cuff noted above, and the stimulation administered to the sympathetic ganglia by the doctor/practitioner would not be necessary because of the percussion from the mat. It should be understood that the mat and the cuff are of conventional design. The present invention is designed to engage the multimodal functioning of the brain primarily through audio signals but also through visual textual signals shown on the monitor of the computer from the software program.

The signal representing the substance is played to the patient via the software program. At the same time, the motors of the mat are activated order to stimulate the sympathetic ganglia to reduce sympathetic nerve activity. It should be understood that the mat of the present invention performs the stimulation, and not the practitioner. An overview of the mat apparatus and overarching system of the present invention and how it is used is outlined below. The components of the work product include, a computer control application, and informational signal delivery methods computer screen, speakers, and mat for stimulation of sympathetic ganglia.

Apparatus Definition—COMPUTER Application

Include a USE driver for the Computer recognition of, and ability to communicate with, the apparatus of the present invention.

The Computer can communicate with only a single apparatus.

No user management system is required. Application does require login or password protection.

A database is required for data storage.

Session/test management
Create a test session
   Test session name (text input)
   Test type (text input)
   Notes/Comments (text input)

Start a test session
Begins/opens the above-created test session
The practitioner selects an agent signals from the agent library. This agent may be presented to the patient in one of several ways.
The practitioner selects agent distribution method
   Audible via Computer
   Visual via Computer
   Audible via Computer and Visual via Computer
   Audible via Mat
   Audible Mat and Visual via Computer
Practitioner sets vibration parameters for Optional Mat:
Practitioner initializes the session
PC application sends “begin session” command to apparatus

Session review

Search for sessions
Open session
   Displays session data (as listed in 5.4.3)
   Save as .csv file
   Print session results (.pdf)
   By date
   By session name
   By test type

Apparatus Definition—Sympathetic Chain Stimulation Mat A mat provides sympathetic chain stimulation and audible transmission of the agent sounds. The mat may be used instead of the aforementioned cuff. During testing, patient will lie on the mat. The test process same as described above with the addition of the stimulation functionality listed below.

1. Mat is 175 cm (68.89 in) long and 62 cm (24.40 in) wide.

2. Mat is 1″ (2.54 cm) foam padding and cotton covering for patient comfort (see FIG. 1). Additional pillows or towels may be used to provide patient maximum comfort and head support.

3. Mat is connected to the control computer via USE connection.

4. Mat has an external power supply to power the vibration motors.

5. Mat is controlled per selections made by the practitioner in the computer application.

6. Mat has a speaker embedded at the upper portion of the mat for audible agent signal transmission

6.1. Speaker is embedded in the foam on the upper portion of the mat (near the head) on the side where control wiring enters the mat

7. Mat has two rows of massage/vibration motors embedded within.

8. Vibration motors are positioned in the mat as follows:
Beginning 35 cm (13.77 in) from the top of the mat
Spaced 3 cm (1.18 in) apart
Extending a total of 60 cm (23.62 in) down the mat
Final 20 cm (20.87 in) of vibration motors are grouped for separate control to enable flexible use for varying sizes of patients.

Practitioner is able to control the vibration motors via the computer application to target the sympathetic ganglia chain. Vibration motors are controllable in 4 separate groups.

Practitioner is able to control the intensity/strength of the vibration motors via the computer application.

Stimulation can be selected to run only for a selected time period during the signal transmission (3-5 seconds), or can be set to run continuously.

It again, should be noted that the stimulation can be administered manually, electrically through TENS, or via the mat.

It should be understood that the present invention is a method of training a living body of a patient, such that the following steps would he performed: positioning a speaker in the proximity of the patient; transmitting, via a computer, representational signals from the speaker, each signal representing a corresponding irritant; and administering the representational signals to the patient. The present invention also calls for stimulating the sympathetic chain when administering the representational signals via the speaker. Also, according to the present invention, one would ensure that converting each signal into a digital format is carried out. Further, the present invention calls for storing and matching each signal with the corresponding offending agent in a computer database.

Additionally, according to the present invention, one would place the speaker which is embedded into a cuff, the cuff configured to secure to an upper extremity of the patient. Moreover, the present invention can be viewed as positioning a speaker in the proximity of the patient; playing a signal from the speaker toward the patient, the signal matched with corresponding irritant; and stimulating the entire sympathetic chain locations while playing the signal from the speaker. Furthermore, the present invention should be viewed as positioning a speaker in the proximity of the patient; be it in within a mat or a cuff, playing, via a computer, signals from the speaker in the proximity of the patient's ears, each signal matched with a corresponding substance; facing the speaker toward the body of the patient; converting each signal into a digital format via a computer; storing and matching each signal with the corresponding substance in a computer database; placing the speaker onto a cuff or matt, the cuff or matt configured to play the signal in the proximity of the patient's ears. It should be noted that stimulation may be administered manually, electrically or via the mat.

It should be understood that the following is part of the present invention:
1. Non-invasive Transcutaneous Stimulation of the Sympathetic Ganglia
   Stimulation of the entire sympathetic ganglia chain (T1-L2) for 5-10 seconds during presentation of conditioned stimulus.
   Motor vibration (mat) PREFERRED EMBODIMENT
   Electrical percussion (any electrical device that elicits percussive stimulation)
   Manual percussion (non-electrical transcutaneous percussive stimulation)
   Transcutaneous electrical nerve stimulation (TENS) PREFERRED EMBODIMENT for non-responsive cases.
      Low frequency (100 Hz/200 μs) applied with self-adhesive electrodes in the paravertrebral ganglionar region (T1-L2)
      Current delivered sensory-level intensity for 5-10 seconds
   Includes any form of stimulation other regions of the body found to decrease activity of the sympathetic nervous system, such as the auricular branch of the vagus nerve.
2. Representation of Offending Agents
   Stimuli presented during stimulation of sympathetic ganglia
   Visual linguistic representations from computer monitor
   Auditory linguistic representations (via cuff or mat—already described) PREFERRED EMBODIMENT
   Actual substance
      Substance presented to the surface of the skin

Includes any form of stimuli that becomes associated with increased sympathetic nerve activity resulting in a pathological or abnormal reaction. This may include, but is not limited to allergies, sensitivities, anxiety disorders, phobias and unwanted behaviors.

I claim:

1. A method of training a living body to not react to substances, comprising:
a computer playing a digitized sound, the sound being a digital audio representation of a substance the living body is inappropriately reacting to;
electrically stimulating the sympathetic ganglia y transcutaneous, electrical nerve stimulation;
forming a new association with the substance and modifying a physiological reaction to the substance by said a computer playing a digitized sound, the sound being a digital audio representation of a substance the living body is inappropriately reacting to, while said electrically stimulating the sympathetic ganglia by transcutaneous, electrical nerve stimulation.

2. The method of claim 1, further comprising providing visual textual signals while said a computer playing a digitized sound, the sound being a digital audio representation of a substance the living body is inappropriately reacting to, while said electrically stimulating the sympathetic ganglia by transcutaneous, electrical nerve stimulation.

3. The method of claim 2, further comprising the computer maintaining a database that breaks substances down into components.

4. The method of claim 3, further comprising a mat providing subcutaneous sympathetic ganglia chain simulation and audible transmission of sound.

5. The method of claim 4, wherein the mat contains vibratory motors designed for stimulation.
